(19) United States
(12) Patent Application Publication
(10) Pub. No.: US 2024/0014694 A1
(43) Pub. Date: Jan. 11, 2024

(54) MOVABLE COIL IN A RECHARGER DEVICE

(71) Applicant: Medtronic, Inc., Minneapolis, MN (US)

(72) Inventors: Andrew Thomas Fried, Saint Paul, MN (US); Robert J. Monson, Roseville, MN (US)

(21) Appl. No.: 18/142,475

(22) Filed: May 2, 2023

Related U.S. Application Data
(60) Provisional application No. 63/358,613, filed on Jul. 6, 2022.

(51) Int. Cl. H02J 50/90; H02J 50/00; H02J 50/80; H02J 50/40; H02J 50/23

(52) U.S. Cl. CPC H02J 50/90; H02J 50/005; H02J 50/80; H02J 50/402; H02J 50/23; A61N 1/3787

(57) ABSTRACT

A charging device is described. One example of a charging device described herein includes at least one coil contained in a housing, where the at least one coil wirelessly transfers energy to an implantable medical device. The charging device may further include a coil manipulator that adjusts a configuration of the at least one coil within the housing.

### CROSS-REFERENCE TO RELATED APPLICATIONS

The present application claims the benefit of and priority to U.S. Provisional Application No. 63/358,613, filed on Jul. 6, 2022, entitled “Movable Coil in a Recharger Device”, which application is incorporated herein by reference in its entirety.

### BACKGROUND

The present disclosure is generally related to energy transfer devices and, in particular, to energy transfer devices that are useable in connection with implantable medical devices.

Implantable medical devices may provide a therapeutic result (e.g., pain management, neurostimulation, etc.) for a patient. Some implantable medical devices may utilize energy delivered or transferred from an external charging device. Improved techniques for charging an implanted medical device are desired.

### BRIEF SUMMARY

Example aspects of the present disclosure include:

A charging device is provided comprising: at least one coil contained in a housing, wherein the at least one coil wirelessly transfers energy to an implantable medical device; and a coil manipulator that adjusts a configuration of the at least one coil within the housing.

In some embodiments, the coil manipulator moves the at least one coil within the housing.

In some embodiments, the coil manipulator moves the at least one coil within the housing by translating a position of the at least one coil within the housing.

In some embodiments, the coil manipulator moves the at least one coil within the housing by rotating a position of the at least one coil within the housing.

In some embodiments, the charging device further includes at least one sensor that provides a sensor reading to a charging engine.

In some embodiments, the charging engine instructs the coil manipulator to adjust the configuration of the at least one coil based on the sensor reading.

In some embodiments, the sensor reading provides an indication of an efficiency with which the at least one coil is wirelessly transferring energy to the implantable medical device.

In some embodiments, the sensor reading provides an indication of a distance between the at least one coil and a receiver coil in the implantable medical device.

In some embodiments, the at least one coil is provided on a support substrate in the housing and wherein the coil manipulator adjusts the configuration of the at least one coil by moving the support substrate.

In some embodiments, the at least one coil comprises a wire mounted on the support substrate.

In some embodiments, the at least one coil comprises a conductive material printed on the support substrate.

In some embodiments, the at least one coil comprises a single coil.

In some embodiments, the at least one coil comprises an array of coils.

In some embodiments, the coil manipulator comprises one or more electrical switches that selectively activate and deactivate coils in the array of coils.

In some embodiments, the configuration of the at least one coil is adjusted to maximize charging efficiency between the at least one coil and the implantable medical device.

A system is also provided, comprising: a chargeable device having a receiver coil; and a charging device comprising: a charging coil that wirelessly transfers energy to the receiver coil; and a coil manipulator that changes a configuration of the charging coil to improve an efficiency with which the energy is wirelessly transferred to the receiver coil during a charging session.

In some embodiments, the system further comprises at least one sensor that provides a sensor reading to determine an efficiency with which the energy is wirelessly transferred to the receiver coil during the charging session.

In some embodiments, the at least one sensor comprises one or more of an electromagnetic sensor, an optical sensor, an acoustic sensor, a mechanical sensor, and a thermal sensor.

In some embodiments, the coil manipulator at least one of physically moves the charging coil or adjusts an RF field produced by the charging coil.

A system is also provided, comprising: a housing; a charging coil contained within the housing; and a coil manipulator that physically moves the charging coil in the housing.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/implementations in combination with any one or more other aspects/features/implementations.

Use of any one or more of the aspects or features as disclosed herein.

It is to be appreciated that any feature described herein can be claimed in combination with any other feature(s) as described herein, regardless of whether the features come from the same described implementation.

The details of one or more aspects of the disclosure are set forth in the accompanying drawings and the description below. Other features, objects, and advantages of the techniques described in this disclosure will be apparent from the description and drawings, and from the claims.

The preceding is a simplified summary of the disclosure to provide an understanding of some aspects of the disclosure. This summary is neither an extensive nor exhaustive overview of the disclosure and its various aspects, implementations, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, implementations, and configurations of the disclosure are possible utilizing, alone or in combination, one or more of the features set forth above or described in detail below.

Numerous additional features and advantages of the present disclosure will become apparent to those skilled in the art upon consideration of the implementation descriptions provided hereinbelow.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, implementations, and configurations of the disclosure, as illustrated by the drawings referenced below.

FIG. 1 is a block diagram of a system according to at least one embodiment of the present disclosure;

FIG. 2 illustrates additional details of a system according to at least one embodiment of the present disclosure;

FIG. 3A illustrates a first configuration of a system according to at least one embodiment of the present disclosure;

FIG. 3B illustrates a second configuration of a system according to at least one embodiment of the present disclosure;

FIG. 3C illustrates a third configuration of a system according to at least one embodiment of the present disclosure;

FIG. 4 is a flow diagram illustrating a charging method according to at least one embodiment of the present disclosure; and

FIG. 5 is a flow diagram illustrating another charging method according to at least one embodiment of the present disclosure.

### DETAILED DESCRIPTION

It should be understood that various aspects disclosed herein may be combined in different combinations than the combinations specifically presented in the description and accompanying drawings. It should also be understood that, depending on the example or implementation, certain acts or events of any of the processes or methods described herein may be performed in a different sequence, and/or may be added, merged, or left out altogether (e.g., all described acts or events may not be necessary to carry out the disclosed techniques according to different implementations of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10X Fusion processors; Apple A11, A12, A12X, A12Z, or A13 Bionic processors; or any other general purpose microprocessors), graphics processing units (e.g., Nvidia GeForce RTX 2000-series processors, Nvidia GeForce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any implementations or embodiments of the disclosure are explained in detail, it is to be understood that the disclosure is not limited in its application to the details of construction and the arrangement of components set forth in the following description or illustrated in the drawings. The disclosure is capable of other implementations and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The terms proximal and distal are used in this disclosure with their conventional medical meanings, proximal being closer to the operator or user of the system, and further from the region of surgical interest in or on the patient, and distal being closer to the region of surgical interest in or on the patient, and further from the operator or user of the system.

In some systems, implanted medical devices (e.g., implanted neurostimulators, neurostimulators having a titanium enclosure, etc.) may provide a therapeutic result (e.g., pain management, neurostimulation, etc.) for a patient. Some implantable medical devices may be wirelessly rechargeable, as the implantable medical devices may include rechargeable power supplies capable of being charged by wireless energy delivered or transferred from an external charging device. Accordingly, for example, the lifetime power utility of a rechargeable device is relatively high relative compared to devices powered by a primary cell (e.g., a single use battery).

Example drawbacks associated with some rechargeable implantable medical devices include a significant burden to patients associated with regular (e.g., daily, periodic, semi-periodic, continuous, etc.) charging of such implantable medical devices. For example, some implantable medical devices may involve charging (recharging) as often as once or twice per day, with each charging session lasting for 1 to 2 hours. For some implantable medical devices with very high energy demands, charging sessions may last 8 hours per day or more (e.g., all day long).

Some external charging device implementations involve a user carrying or wearing a charging device (e.g., a puck-shaped wireless charging device, an antenna connected to a cable, etc.) while charging a corresponding implantable medical device. Accordingly, for example, improved efficiency with respect to charging (e.g., charging duration, charging frequency, etc.) are desired. Additionally, some external charging device implementations may include temporary fixation devices (e.g., a belt or strap, a wearable having a holster, a holder, etc.) to temporarily maintain a position of the charging device proximal to the implantable medical device (e.g., maintain a position of an external coil thereof proximal to the implantable medical device). Accordingly, for example, devices that enhance charging efficiencies or reduce the amount of time that a user has to maintain the charging device proximal to the implantable medical device can vastly improve user experience.

According to example aspects of the present disclosure, a charging device (also referred to herein as a recharging device or a recharger) capable of charging and/or recharging an implantable medical device is described. The implantable medical device may be an implantable neurostimulator (INS) implanted in a subject (e.g., a patient).

When a charging device is placed against or near the skin of a patient and in proximity with an implanted device, the recharge process can be conducted. Alignment of the coil in the charging device and the receiving coil in the implanted device is an important variable when it comes to charging efficiency (e.g., the amount of energy transferred from the charging device to the implanted device). Embodiments of the present disclosure contemplate that the coil in the charging device can be configured to float within a housing of the charging device, meaning that a position or orientation of the charging coil can be adjustable. In some embodiments, the coil may be moveable within the housing and may have its position adjusted by microcontrollers on a real-time basis during a recharge process. In some embodiments, the coil may be repositioned in an attempt to improve recharge efficiencies and to reduce an amount of time that the patient has to undergo the recharge process. Moving the coil in the charging device is quite different from existing charging systems, which propose to move the device being charged, but such movements may not be possible when charging an implanted device. Moreover, if the patient has already temporarily fixed a position of the charging device relative to the implant (e.g., because the charging device is held in place by a temporary fixation device), then gross movements of the charging device may not be possible without user involvement, but it still may be possible to adjust a position of the coil within the housing of the charging device to improve charging efficiencies. This minimizes the amount of adjustment required by the patient, which may also improve user experience.

During the recharge cycle, the implantable device may be configured to provide feedback related to recharge efficiency or recharge efficiency may be measured inherently by monitoring other characteristics of the recharge process. In some embodiments, based on feedback or measured characteristics, the charging device may make fine adjustments to the coil position within the housing of the charging device to improve recharge efficiency.

Aspects of the present disclosure will be described with respect to charging systems in which a charging device wirelessly transfers energy (e.g., charging, recharging) to an implanted medical device. While embodiments will be described with respect to charging implanted medical devices, it should be appreciated that the concepts disclosed herein can be applied to the charging of non-implanted devices and non-medical devices. For instance, embodiments of the present disclosure may be applied to charging systems for wearable (and removable) devices, smartphones, laptops, etc. Indeed, features of the present disclosure should not be construed as being limited to implantable devices and/or medical devices, but rather can be applied to any system that wirelessly charges a chargeable device with a charging device.

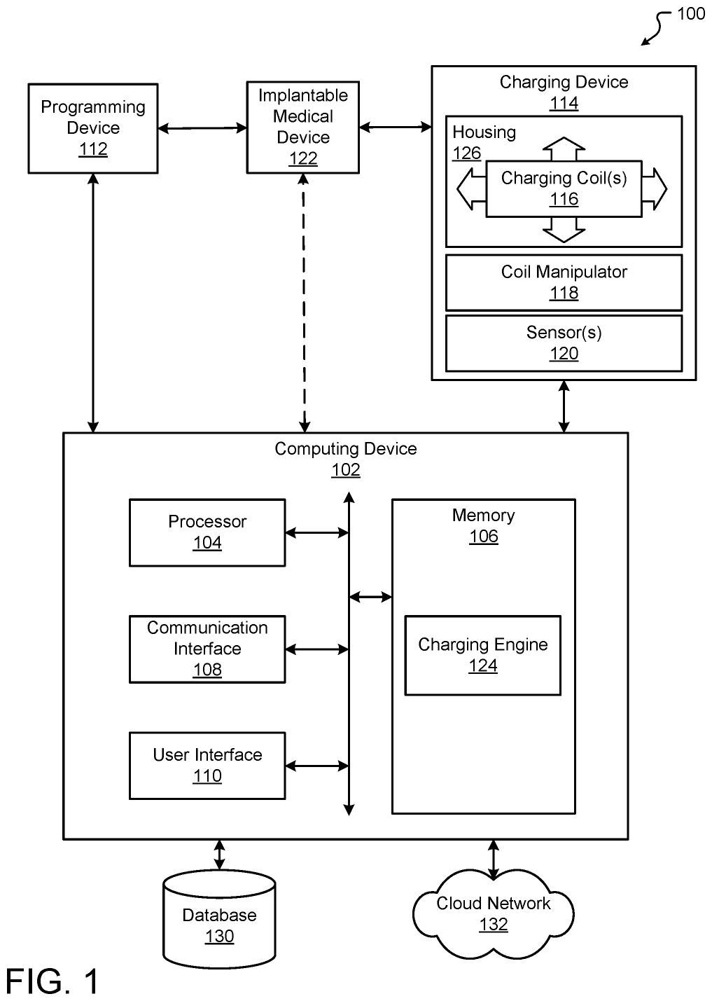

FIG. 1 illustrates an example of a block diagram of a system 100 according to at least one implementation of the present disclosure.

The system 100 includes a computing device 102, a programming device 112, a charging device 114, an implantable medical device 122, a database 130, and/or a cloud network 132 (or other network). Systems according to other implementations of the present disclosure may include more or fewer components than the system 100. For example, the system 100 may omit and/or include additional instances of the programming device 112, the charging device 114, the implantable medical device 122, one or more components of the computing device 102, the database 130, and/or the cloud 132. The system 100 may support the implementation of one or more other aspects of one or more of the methods disclosed herein.

According to example aspects of the present disclosure, the charging device 114 may be capable of wirelessly charging and/or recharging the implantable medical device 122, which may be referred to as a chargeable device. The charging device 114 may include a housing 126 in which one or more charging coils 116 are contained. The charging device 114 may also include a coil manipulator 118 and one or more sensors 120. As will be described in further detail herein, the coil manipulator 116 may include hardware and/or software that are capable of physically moving, rotating, or adjusting the charging coil(s) 116 within the housing 126 of the charging device 114. In some embodiments, the coil manipulator 116 may employ data (e.g., instructions and/or Artificial Intelligence (AI) models) that is contained within the coil manipulator 118. Alternatively or additionally, the coil manipulator 118 may correspond to the physical elements that adjust a position of the charging coil(s) 116, but the coil manipulator 118 may be operated with a charging engine 124 provided in the computing device 102.

The computing device 102 includes a processor 104, a memory 106, a communication interface 108, and a user interface 110. Computing devices according to other implementations of the present disclosure may include more or fewer components than the computing device 102. The computing device 102 may be, for example, a control device including electronic circuitry associated with driving a charging coil 116 of the charging device 114. As mentioned above, the computing device 102 may also include a charging engine 124 that provides logic to control a position of the charging coil(s) 116 relative to the implantable medical device 122. The charging engine 124 may be used to determine improved positioning of the charging coil(s) 116 and to provide control signals to the coil manipulator 118 that cause the coil manipulator 118 to adjust a position of the charging coil(s) 116.

Non-limiting examples of the computing device 102 may include, for example, personal computing devices or mobile computing devices (e.g., handsets, tablets, wearable devices, mobile phones, smart phones, smart devices, etc.). In some examples, the computing device 102 may be operable by or carried by a human user. In some embodiments, the computing device 102 may perform one or more operations autonomously or in combination with an input by the user. The processor 104 of the computing device 102 may be any processor described herein or any similar processor. The processor 104 may be configured to execute instructions stored in the memory 106, which instructions may cause the processor 104 to carry out one or more computing steps utilizing or based on data received from the programming device 112, the charging device 114, the implantable medical device 122, the database 130, and/or the cloud 132.

The memory 106 may be or include RAM, DRAM, SDRAM, other solid-state memory, any memory described herein, or any other tangible, non-transitory memory for storing computer-readable data and/or instructions. The memory 106 may store information or data associated with completing, for example, any step of any method described herein. The memory 106 may store, for example, instructions and/or machine learning models that support one or more functions of the programming device 112, the computing device 102, and/or the charging device 114. For instance, the memory 106 may store content (e.g., instructions and/or machine learning models) that, when executed by the processor 104, enable device charging (e.g., by a charging engine 124) and/or enable manipulation of the charging coil(s) 116 via the coil manipulator 118. Thus, the charging engine 124, when executed by the processor 104, may enable the computing device 102 to cooperate with the charging device 114 for purposes of efficiently charging the implantable medical device 122. Such content, if provided as in instruction, may, in some implementations, be organized into one or more applications, modules, packages, layers, or engines. Alternatively or additionally, the memory 106 may store other types of content or data (e.g., machine learning models, artificial neural networks, deep neural networks, etc.) that can be processed by the processor 104 to carry out the various method and features described herein. Thus, although various contents of memory 106 may be described as instructions, it should be appreciated that functionality described herein can be achieved through use of instructions, algorithms, and/or machine learning models. The data, algorithms, and/or instructions may cause the processor 104 to manipulate data stored in the memory 106 and/or received from or via the programming device 112, the charging device 114, the implantable medical device 122, the database 130, and/or the cloud 132. Aspects described herein of the charging engine 124 may be implemented at the computing device 102, the programming device 112, and/or the charging device 114. It should be appreciated that while the computing device 102 is depicted as being separate from the charging device 114, the components of the computing device 102 and charging device 114 may be provided in a single device without departing from the scope of the present disclosure.

The computing device 102 may also include a communication interface 108. Alternatively or additionally, the computing device 102 may be combined with the charging device 114. The communication interface 108 may be used for receiving data or other information from an external source (e.g., the programming device 112, the charging device 114, the implantable medical device 122, the database 130, the cloud 132, and/or any other system or component separate from the system 100), and/or for transmitting instructions, data (e.g., measurements, sensor readings, etc.), or other information to an external system or device (e.g., another computing device 102, the programming device 112, the charging device 114, the implantable medical device 122, the database 130, the cloud 134, and/or any other system or component not part of the system 100). The communication interface 108 may include one or more wired interfaces (e.g., a USB port, an Ethernet port, a Firewire port) and/or one or more wireless transceivers or interfaces (configured, for example, to transmit and/or receive information via one or more wireless communication protocols such as 802.11a/b/g/n, Bluetooth, NFC, ZigBee, and so forth). In some implementations, the communication interface 108 may support communication between the computing device 102 and one or more other processors 104 or computing devices 102, whether to reduce the time needed to accomplish a computing-intensive task or for any other reason.

In some aspects, the computing device 102 may support direct and indirect communications with any of the programming device 112, the charging device 114, the implantable medical device 122, the database 130, the cloud 134, and/or any other system or component separate from the system 100. In an example, the computing device 102 may communicate directly (e.g., using wireless communications) with the implantable medical device 122. In another example, the computing device 102 may communicate indirectly with the implantable medical device 122 (e.g., via the charging device 114). In some implementations, the computing device 102 may be combined with the charging device 114 in the same assembly.

The computing device 102 may also include one or more user interfaces 110. The user interface 110 may be or include a keyboard, mouse, trackball, monitor, television, screen, touchscreen, and/or any other device for receiving information from a user and/or for providing information to a user. The user interface 110 may be used, for example, to receive a user selection or other user input regarding any step of any method described herein. Notwithstanding the foregoing, any required input for any step of any method described herein may be generated automatically by the system 100 (e.g., by the processor 104 or another component of the system 100) or received by the system 100 from a source external to the system 100. In some implementations, the user interface 110 may support user modification (e.g., by a surgeon, medical personnel, a patient, etc.) of instructions to be executed by the processor 104 according to one or more implementations of the present disclosure, and/or to user modification or adjustment of a setting of other information displayed on the user interface 110 or corresponding thereto.

In some implementations, the computing device 102 may utilize a user interface 110 that is housed separately from one or more remaining components of the computing device 102. In some implementations, the user interface 110 may be located proximate one or more other components of the computing device 102, while in other implementations, the user interface 110 may be located remotely from one or more other components of the computer device 102.

The programming device 112 may program operations associated with the implantable medical device 122. The programming device 112 may include some of the components of the computing device 102 described herein. For example, the programming device 112 may include a processor, a communication interface, memory, content (e.g., executable instructions and/or machine learning models that enable programming a control device), a user interface, or the like described herein.

The charging device 114 (also referred to herein as a recharging device or a recharger) may be capable of charging and/or recharging the implantable medical device 122 (also referred to herein as a rechargeable device). Additionally, or alternatively, the charging device 114 may be capable of charging devices (e.g., external medical devices, external neurostimulation devices, wearable devices, personal communication devices, portable computing devices, etc.) other than the implantable medical device 122. The charging device 114 may include one or more charging coils 116, a coil manipulator 118, one or more transmitters, and one or more sensors 120. In some embodiments, the charging coil(s) 116 may be maintained within a housing of the charging device 114, thereby limiting exposure of the charging coil(s) 116 to environmental hazards, debris, or the like. As will be discussed in further detail herein, the charging coil(s) 116 may be movable (e.g., via translation and/or rotation) within the housing 126. The coil manipulator may include mechanical and/or software components that are capable of moving the charging coil(s) 116 within the housing 126. In some embodiments, the coil manipulator 118 may respond to instructions issued by the charging engine 124 and may physically manipulate the charging coil(s) 116 based on such instructions. Accordingly, the coil manipulator 118 may include one or more of a servo motor, stepper motor, rotatable plate, translatable plate, mechanical actuator, mechanical hinge, or combinations thereof. A relative size of the housing 126 and the charging coil(s) 116 may limit an amount of motion that can be imparted on the charging coil(s) 116. Said another way, motion of the charging coil(s) 116 may be limited by the boundaries or extent of the housing 126.

In some embodiments, the sensor(s) 120 may be used to measure information about the charging coil(s) 116, an environment surrounding the charging coil(s) 116, a position of the charging coil(s) 116 within the housing 126, or the like. Sensor(s) 120 of the implantable medical device 122 may cooperate with sensors of the charging device 114 to determine a relative position of the two devices. Information obtained from the sensor(s) 120 may be used to control the coil manipulator 118. For example, readings from the sensor(s) 120 may be provided to the charging engine 124, which adjusts a control signal transmitted to the coil manipulator 118 based on the sensor readings. Thus, a feedback loop may be created in which sensor readings are used to adjust, maintain, or optimize a position of the charging coil(s) 116 within the housing 126. In some embodiments, the sensor(s) 120 may be used to measure a charging efficiency and then the charging engine 124 may attempt to improve the charging efficiency (as measured by the sensor(s) 120) by adjusting a position of the charging coil(s) 116 with the coil manipulator 118. Non-limiting examples of sensor(s) 120 that may be used to measure charging efficiency or other characteristics of the charging device 114 and/or implantable medical device 122 during a charging process include electromagnetic sensors (e.g., hall sensors, capacitive sensors, inductive sensors, current sensors, etc.), optical sensors (e.g., cameras, Infrared sensors, proximity sensors, light sensors, etc.), acoustic sensors (e.g., ultrasound sensors, microphones, diaphragms, etc.), mechanical sensors (e.g., haptic sensors, accelerometers, pressure sensors, strain gauges, etc.), thermal sensors (e.g., temperature sensors, heat sensors, etc.), sensor arrays, or combinations thereof. As noted herein, sensor(s) 120 may alternatively or additionally be included in the implantable medical device 122, which may be configured to work independently or in cooperation with components of the charging device 114. It should be noted that a transmitter may also be needed for certain sensors. For example, an ultrasound transmitter or LED could be provided as a transmitter/sensor on the charging device 114 and/or. Transmitter/sensor could be on either 114 or implantable medical device 122. More specifically, but without limitation, a white or infrared LED may be provided in the implantable medical device 122 beneath a sapphire window and a phototransistor may be provided in the charging device 114, behind an optically transparent window or light pipe.

Because wireless charging may be used by the charging device 114, one or more sensors 120 may be configured to measure electrical characteristics of the charging coils 116 (e.g., current, induction, power, etc.) and/or conditions surrounding the coil(s) 116 (e.g., RF field strength, magnetic field strength, RF field distribution, magnetic field distribution, etc.). One or more measurements obtained by the sensor(s) 120 may be used to characterize an efficiency with which the charging device 114 is delivering charge to the implantable medical device 122. It should be appreciated that higher charging efficiency may result in decreased charging time(s), which may be desirable from a patient perspective.

In some examples, the charging device 114 may include aspects of the computing device 102. For example, the charging device 114 may also include processors, memory, a communications interface, a user interface, or the like as described with reference to the computing device 102. The charging device 114 may include one or more primary recharge antennas for transferring power to the implantable medical device 122. It is possible that the charging coil(s) 116 may include two, three, four, or more coils or an array of coils that are selectively activated/deactivated to focus power of the charging coils 116 onto receiver coils provided in the implantable medical device 122. The charging engine 124 may determine which of the plurality of coils in the charging coil(s) 116 should be activated to maximize an efficiency with which the implantable medical device 122 is charged. In such a scenario, the coil manipulator 118 may not necessarily require a mechanical actuator, but rather may include one or more switching circuits that selectively activate/deactivate particular coils in the plurality of coils.

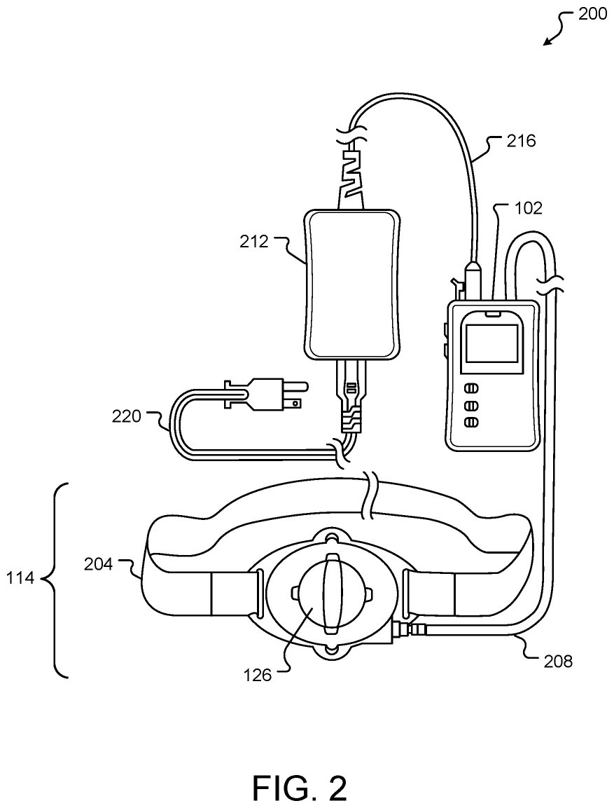

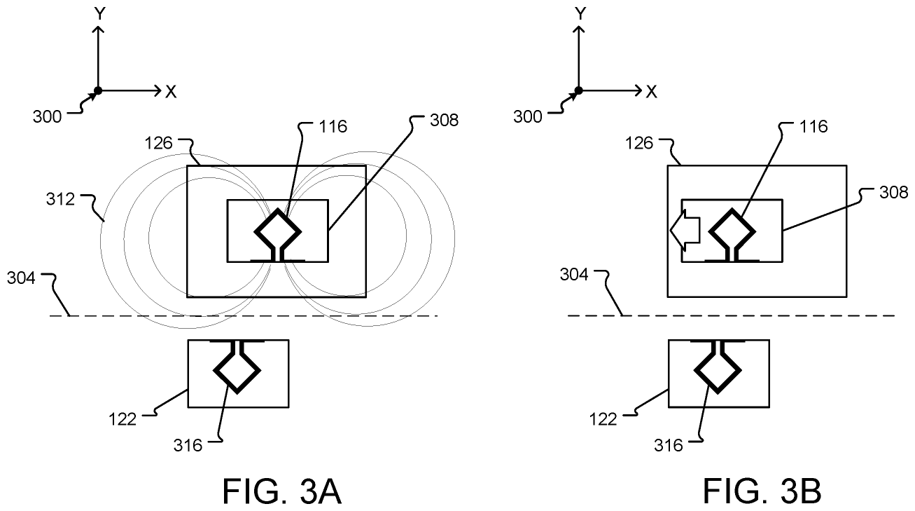

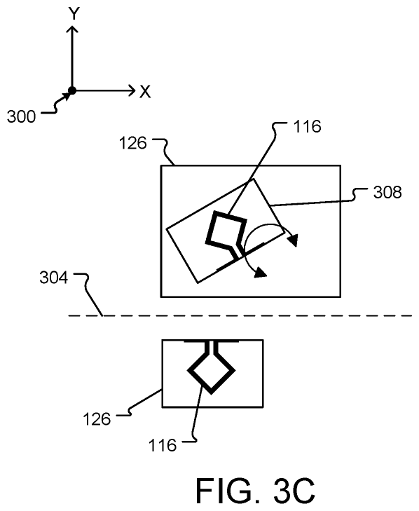

The implantable medical device 122 may be, for example, an implanted neurostimulator (also referred to herein as a neurostimulator device) implantable in a patient. The implantable medical device 122 may include one or more of the sensors depicted and/or described herein (e.g., electromagnetic sensor, an optical sensor, an acoustic sensor, a mechanical sensor, and a thermal sensor). The implantable medical device 122 may deliver neurostimulation therapy to a patient, for example, via one or more leads that include electrodes positioned or located proximate to an anatomical element (e.g., spinal cord, nerves, stomach, brain, etc.) of a patient. Alternatively or additionally, the implantable medical device 122 may include a leadless device that has electrodes on the surface of the enclosure. The implantable medical device 122 may deliver neurostimulation therapy in the form of electrical pulses. Additional aspects of the implantable medical device 122 are later described herein with reference to FIGS. 2 and 3.

The charging engine 124 may support control of charging operations described herein. For example, the charging engine 124 may control and/or set any combination of parameters (e.g., duration, start time, stop time, scheduling, intensity, target power, temperature setpoint, etc.) associated with the charging operations. In some aspects, the charging engine 124 may control initiating, pausing, modifying, and/or terminating a charging session.

The charging engine 124 may support monitoring of temperatures of the charging device 114 (e.g., one or more components thereof) or the implantable medical device 122 or both. For example, the charging engine 124 may monitor or measure temperature values of components of the charging device 114, may measure power generated by the charging device 114, may measure current provided to the charging device 114, etc. In some aspects, the charging engine 124 may monitor or measure temperature values at points of contact between the charging device 114 and the patient. In some aspects, the charging engine 124 may control the output of notifications (e.g., haptic, visual, audio, etc.) to the user.

The memory 106 (and/or database 130) may store, for example, data associated with one or more charging sessions. For example, the memory 106 (and/or database 130) may include real-time and/or historical temperature information (e.g., temperature values, rates of temperature increase, etc.) described herein of the charging device 114 in association with one or more charging sessions. The memory 106 (and/or database 130) may store temporal information (e.g., date and/or time of installation) and/or motion information for the charging coil(s) 116.

In some aspects, the memory 106 may store machine learning models that support predicting when to move or adjust a position of charging coil(s) 116 within the housing during a charging session. For example, the machine learning models may support testing different positions or configurations of the charging coil(s) 116 in an attempt to improve charging efficiency.

The database 130 may store information that correlates one coordinate system to another. The database 130 may additionally or alternatively store, for example, location or coordinates of the implantable medical device 122, positions of charging coil(s) 116, and the like. The database 130 may be configured to provide any such information to the computing device 102 or to any other device of the system 100 or external to the system 100, whether directly or via the cloud 132. In some implementations, the database 130 may include treatment information (e.g., a pain management plan) associated with a patient. In some implementations, the database 130 may be or include part of a hospital image storage system, such as a picture archiving and communication system (PACS), a health information system (HIS), and/or another system for collecting, storing, managing, and/or transmitting electronic medical records including image data.

In some aspects, the computing device 102 may communicate with a server(s) and/or a database (e.g., database 130) directly or indirectly over a communications network (e.g., the cloud network 132). The communications network may include any type of known communication medium or collection of communication media and may use any type of protocols to transport data between endpoints. The communications network may include wired communications technologies, wireless communications technologies, or any combination thereof.

Wired communications technologies may include, for example, Ethernet-based wired local area network (LAN) connections using physical transmission mediums (e.g., coaxial cable, copper cable/wire, fiber-optic cable, etc.). Wireless communications technologies may include, for example, cellular or cellular data connections and protocols (e.g., digital cellular, personal communications service (PCS), cellular digital packet data (CDPD), general packet radio service (GPRS), enhanced data rates for global system for mobile communications (GSM) evolution (EDGE), code division multiple access (CDMA), single-carrier radio transmission technology (1×RTT), evolution-data optimized (EVDO), high speed packet access (HSPA), universal mobile telecommunications service (UMTS), 3G, long term evolution (LTE), 4G, and/or 5G, etc.), Bluetooth®, Bluetooth® low energy, Wi-Fi, radio, satellite, infrared connections, and/or ZigBee® communication protocols.

The Internet is an example of the communications network that constitutes an Internet Protocol (IP) network consisting of multiple computers, computing networks, and other communication devices located in multiple locations, and components in the communications network (e.g., computers, computing networks, communication devices) may be connected through one or more telephone systems and other means. Other examples of the communications network may include, without limitation, a standard Plain Old Telephone System (POTS), an Integrated Services Digital Network (ISDN), the Public Switched Telephone Network (PSTN), a Local Area Network (LAN), a Wide Area Network (WAN), a wireless LAN (WLAN), a Session Initiation Protocol (SIP) network, a Voice over Internet Protocol (VoIP) network, a cellular network, and any other type of packet-switched or circuit-switched network known in the art. In some cases, the communications network 120 may include of any combination of networks or network types. In some aspects, the communications network may include any combination of communication mediums such as coaxial cable, copper cable/wire, fiber-optic cable, or antennas for communicating data (e.g., transmitting/receiving data).

The computing device 102 may be connected to the cloud network 132 via the communication interface 108, using a wired connection, a wireless connection, or both. In some implementations, the computing device 102 may communicate with the database 130 and/or an external device (e.g., a computing device) via the cloud network 132.

The system 100 or similar systems may be used, for example, to carry out one or more aspects of the methods described herein. The system 100 or similar systems may also be used for other purposes.

FIG. 2 illustrates additional details of a system 200, which may be similar to or include similar components as the system 100. The computing device 102 may receive AC power from a power source (not illustrated) via a cable 216, 220 and a transformer 212. In some aspects, the computing device 102 may include rechargeable power source (not illustrated), such as a battery. The computing device 102 may provide power (e.g., via the charging device 114) to the implantable medical device 122 from the power source and/or the rechargeable power source.

The user interface 110 may support controls (not illustrated) associated with starting, pausing, modifying, or stopping a therapy session and/or a charging session (e.g., based on implementation). In some aspects, the user interface 110 may support controls (e.g., a “Start Charge” button, a “Stop Charge” button, and a “Silence” button) associated with starting, pausing, modifying, or stopping a therapy session and/or a charging session. The “Start Charge” button and the “Stop Charge” button may support starting and stopping of a therapy session and/or a charging session. The “Silence” button may support disabling and/or re-enabling of notifications (e.g., video, audio, and/or haptic) by the computing device 102 in association with a therapy session and/or a charging session.

The housing 126 of the charging device 114 may be positioned (e.g., held in a position) with respect to a patient using a wearable element 204. In an example, the wearable element 204 may be a belt, a band, a strap, an item of clothing (e.g., a shirt, pants, a jacket, an undergarment, etc.) or the like. In some other aspects, the wearable element 204 may include a receptacle for holding the charging device 114. The wearable element 204 may be adjustable based on one or more dimensions of the user. In some aspects, the wearable element 204 may be manufactured of any combination of fabrics, flexible materials (e.g., rubber, plastic, etc.), elastic materials, and rigid materials. In some aspects, the charging device 114 may be separate from the wearable element 204. In some other aspects, the charging device 114 may be integrated with the wearable element 204. Illustratively, a data connector 208 may be used to interconnect the computing device 102 with the charging device 114. Sensor readings may pass from the charging device 114 to the computing device 102 via the cable 208. Likewise, control signals, power, and/or current for driving the charging coil(s) 116 may pass from the computing device 102 to the charging device 114 via the cable 208. Thus, the cable 208 may be configured to carry data and/or power.

FIGS. 3A-3C illustrate additional examples of a charging device 114 and the manipulation of charging coil(s) 116 within a housing 126 of the charging device 114 in accordance with at least some embodiments of the present disclosure.

Referring initially to FIG. 3A, the charging coil 116 is shown to be contained within a housing 126 and is situated on a support substrate 308. The charging coil 116 may correspond to a coil of conductive wire that is placed on the support substrate 308 in a loop configuration. Alternatively, the charging coil 116 may correspond to a conductive material that has been printed on the support substrate 308. Regardless of the configuration, the charging coil 116 may be configured to generate an RF field 312 that is capable of delivering wireless power a receiver coil 316 in the implantable medical device 122. As can be appreciated, placement of the receiver coil 316 in a stronger portion of the RF field 312 may help improve charging efficiencies during a charging session. It should be appreciated that coils come in many shapes and sizes. The symbol used for charging coil 116 is not an indication of the coil type or orientation and the coil may be pancake shape parallel to non-contact boundary 304 or cylindrical type aligned with Z axis, could be square, circular, elliptical, etc.

Features of the system 100, 200 can be described in conjunction with a coordinate system 300. The illustrated coordinate system 300 includes three-dimensions comprising an X-axis, a Y-axis, and a Z-axis (not illustrated). Additionally or alternatively, the coordinate system 300 may be used to define planes (e.g., the XY-plane, the XZ-plane, and the YZ-plane) of the system 100, 200. These planes may be disposed orthogonal, or at 90 degrees, to one another. While the origin of the coordinate system 300 may be placed at any point on or near the components of the system 100, 200, for the purposes of description, the axes of the coordinate system 300 are disposed along the same directions from figure to figure, whether the coordinate system 300 is shown or not. In some examples, reference may be made to dimensions, angles, directions, relative positions, and/or movements associated with one or more components of the system 100, 200 with respect to the coordinate system 300.

As shown in FIG. 3A, a position of the implantable medical device 122 may be substantially fixed (e.g., by virtue of being implanted in a patient) and it may not always be possible to perfectly align the housing 126 of the charging device 114 with the implantable medical device 122. Moreover, a non-contact boundary 304 may be maintained between the charging device 114 and the implantable medical device 122 because the implantable medical device 122 may be implanted below the skin of the patient. Thus, it may be necessary to deliver RF energy from the charging coil(s) 116 to the receiver coil 316, where the RF energy passes through the non-contact boundary 304.

As can be seen in FIG. 3B, it may be possible to improve an alignment between the charging coil 116 and the receiver coil 316 by moving the charging coil 116 within the housing 126 of the charging device 114. In the embodiment of FIG. 3B, the charging coil 116 is moved by translating a position of the support substrate 308 within the housing 126 (e.g., in the x-direction or in the XY plane). In some embodiments, it may be desirable to aim toward a shallowest/shortest y-dimension in the image depicted. As mentioned above, the charging coil 116 may be moved by the coil manipulator 118, which may have one or more motor that physically move the support substrate 308 within the housing 126. When comparing the orientation of the system in FIG. 3A with the orientation of the system in FIG. 3B, it can be seen that the charging coil 116 is nearer to the receiver coil 316 after the support substrate 308 has been moved. This improvement in physical proximity between the coils 116, 316 may result in an improved charging efficiency because the receiver coil 316 is placed at a location where the RF field 312 is stronger.

While translation of the charging coil 116 provides one way for improving charging efficiency, other types of motions or manipulations are possible to improve charging efficiency. For instance and as shown with respect to FIG. 3C, the charging coil 116 may be rotated within the housing 126. Rotation of the charging coil 116 may cause the RF field 312 to be adjusted, thereby placing a stronger portion of the RF field 312 over the receiver coil 316. As with the translation example, the charging coil 116 may be moved/rotated within the housing 126 by rotating the support substrate 308 on which the charging coil 316 is placed. Rotation may also be in the XZ plane as well if a cylindrical coil were utilized. For example, a cylindrical coil with its main axis going into and out of the z-direction may rotate around the y-axis if the charging coil 116 is also a cylindrical coil rotated around the y-axis.

Although FIGS. 3B and 3C illustrate physical manipulations of the charging coil 116, it should be appreciated that other manipulations of the charging coil 116 are also contemplated to help improve charging efficiencies. For instance, the RF field 312 may be directed or adjusted by moving objects around the charging coil 116. In other words, the RF field 312 may be manipulated without physically moving the charging coil 116 itself. It may also be possible to induce adjustments to the RF field 312 by selectively activating and/or deactivating one or more charging coils if the charging coils are provided as an array of coils laid out on the support substrate 308. It may also be beneficial to move the charging coils 116 in the y-direction or z-direction if such motion reduces the distance between the charging coil 116 and the receiver coil 316. In some embodiments, readings from sensor(s) 120 may be used to determine if motion of the charging coil 116 will move the charging coil 116 closer to the receiver coil 316. It could also be possible to have two coils oriented in the z-direction and translation may occur in the x-direction.

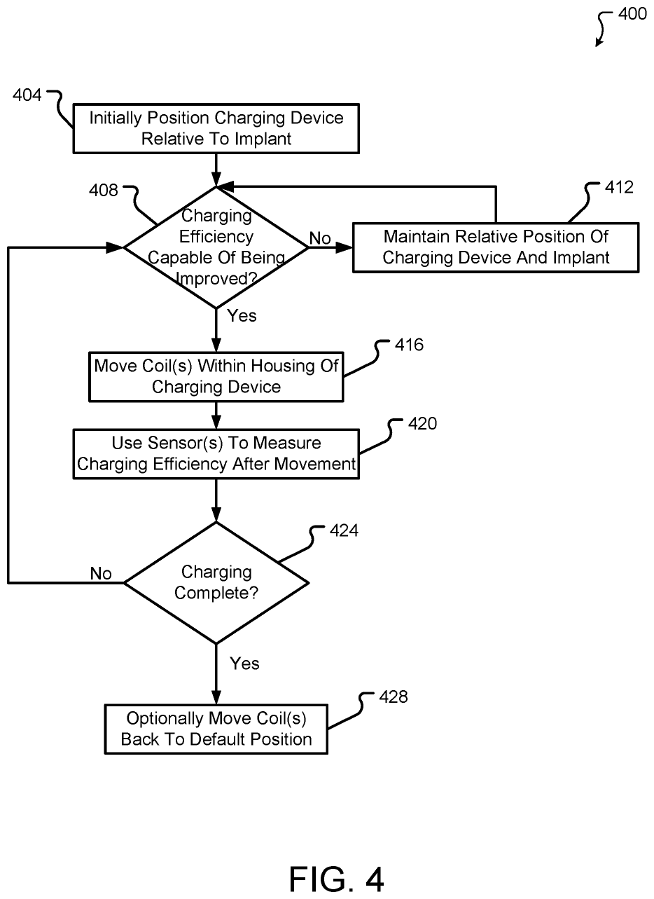

FIG. 4 illustrates an example of a charging method 400 in accordance with aspects of the present disclosure. In some examples, method 400 may implement aspects of a computing device 102, programming device 112, and/or charging device 114 described with reference to FIGS. 1 through 3C. It is to be understood that any of the operations of method 400 may be performed by any device (e.g., a computing device 102, a programming device 112, a charging device 114, etc.).

In the following description of the method 400, the operations may be performed in a different order than the order shown, or the operations may be performed in different orders or at different times. Certain operations may also be left out of the method 400, some operations may be performed simultaneous with one another, or other operations may be added to the method 400.

The method 400 may begin when a charging device 114 is initially positioned with respect to an implantable medical device 122 (step 404). The charging device 114 may be positioned by way of a wearable element 204, which may be worn by a patient. Alternatively or additionally, the patient may hold the charging device 114 on their body and in proximity to the implantable medical device 122. The initial positioning of the charging device 114 may correspond to a gross positioning where the charging coil 116 is positioned relative to the receiver coil 316 with a first distance therebetween.

The method 400 may continue by determining whether the charging efficiency can be improved (step 408). This query may be answered by receiving one or more readings from sensor(s) 120 that characterize a current charging efficiency that is being achieved based on the initial positioning of the charging device 114. If the current charging efficiency as measured by the sensor readings is at or near an expected maximum charging efficiency, then the query of step 408 may be answered negatively. Another approach for answering the query of step 408 is to iteratively move the charging coil(s) 116 within the housing 126 and measure, after each successive movement, the charging efficiency 114 based on the new position of the charging coil(s) 116. If certain movements of the charging coil(s) 116 result in improved charging efficiencies, then the query of step 408 may be answered positively because the initial position of the charging coil(s) 116 did not result in the greatest charging efficiency.

If the query of step 408 is answered negatively, then the initial position of the charging coil(s) 116 is maintained relative to the implantable medical device 122 (step 412). The method 400 may continue by reverting back to step 408 to continue checking if charging efficiencies can be improved during the charging session.

If the query of step 408 is answered positively, then the method 400 may continue by moving the charging coil(s) 116 within the housing of the charging device 126 (step 416). After the charging coil(s) 116 have been moved, the charging efficiency may be measured again (step 420). The method 400 may also determine if the charging session has been completed (step 424). If the charging session has not been completed (e.g., the implantable medical device 122 is not yet fully recharged), then the method 400 may return to step 408 to determine if charging efficiencies can be improved. If the charging session has completed, then the method 400 may optionally continue by moving the charging coil(s) 116 back to a default position within the housing 126 (step 428).

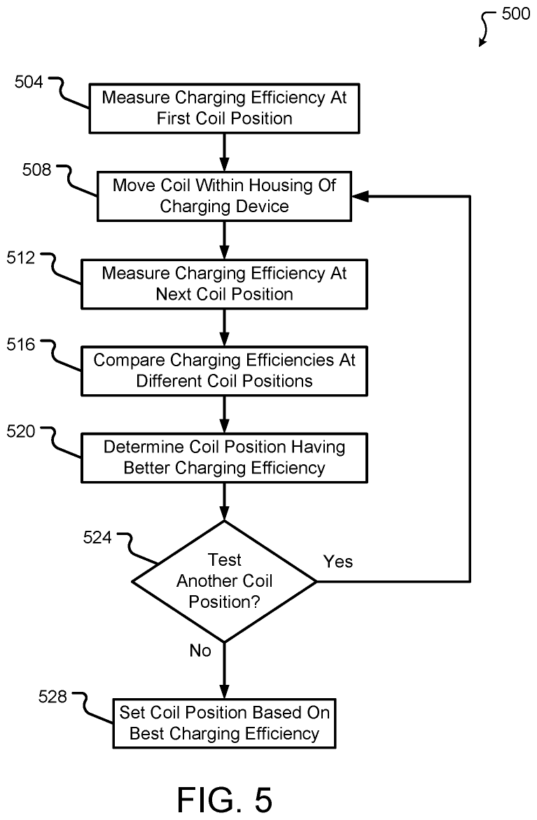

Referring now to FIG. 5, additional details of another charging method 500 will be described in accordance with at least some embodiments of the present disclosure. It should be appreciated that some or all of the steps/operations described with respect to method 500 can be combined or interchanged with various steps/operations described with respect to method 400.

The method 500 begins by measuring a charging efficiency of an implantable medical device 122 when a charging coil 116 is in a first position (step 504). The charging efficiency may be measured or evaluated in a number of different ways as described herein. Specifically, characteristics of the charging device 114, characteristics of the implantable medical device 122, and/or characteristics of an environment surrounding the charging device 114 may be used to measure or determine a charging efficiency when the charging coil 116 is in a first position.

The method 500 may continue with a coil manipulator 118 moving the charging coil 116 within the housing 126 of the charging device 114 (step 508). After the charging coil 116 has been moved, the method 500 may continue by measuring a charging efficiency at the next coil position (step 512). The charging efficiency at the first coil position may be compared with the charging efficiency at the next coil position (e.g., after movement of the charging coil 116) (step 516).

The method 500 may then include determining which coil position resulted in a better charging efficiency (step 520). The method 500 may then include determining whether to test the charging coil 116 at another position (step 524). If the query of step 524 is answered positively, then the charging coil 116 may be moved again and another charging efficiency of the charging coil 116 may be measured (repeating steps 508, 512, 516, 520). If the query of step 524 is answered negatively, then the charging coil 116 may be moved to a position in which the best charging efficiency was measured (step 528). The charging method 500 may help to continually improve charging efficiencies by testing different positions of the charging coil(s) 116 within the housing 126.

It is to be appreciated that any feature described herein can be claimed in combination with any other feature(s) as described herein, regardless of whether the features come from the same described implementation.

The phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” “A, B, and/or C,” and “A, B, or C” means A alone, B alone, C alone, A and B together, A and C together, B and C together, or A, B and C together.

The term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

The term “automatic” and variations thereof, as used herein, refers to any process or operation, which is typically continuous or semi-continuous, done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material.”

Aspects of the present disclosure may take the form of an implementation that is entirely hardware, an implementation that is entirely software (including firmware, resident software, micro-code, etc.) or an implementation combining software and hardware aspects that may all generally be referred to herein as a “circuit,” “module,” or “system.” Any combination of one or more computer-readable medium(s) may be utilized. The computer-readable medium may be a computer-readable signal medium or a computer-readable storage medium.

A computer-readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer-readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer-readable storage medium may be any tangible medium that can contain or store a program for use by or in connection with an instruction execution system, apparatus, or device.

A computer-readable signal medium may include a propagated data signal with computer-readable program code embodied therein, for example, in baseband or as part of a carrier wave. Such a propagated signal may take any of a variety of forms, including, but not limited to, electromagnetic, optical, or any suitable combination thereof. A computer-readable signal medium may be any computer-readable medium that is not a computer-readable storage medium and that can communicate, propagate, or transport a program for use by or in connection with an instruction execution system, apparatus, or device. Program code embodied on a computer-readable medium may be transmitted using any appropriate medium, including, but not limited to, wireless, wireline, optical fiber cable, RF, etc., or any suitable combination of the foregoing.

The terms “determine,” “calculate,” “compute,” and variations thereof, as used herein, are used interchangeably and include any type of methodology, process, mathematical operation or technique.

What is claimed is:

1. A charging device comprising:
at least one coil contained in a housing, wherein the at least one coil wirelessly transfers energy to an implantable medical device; and
a coil manipulator that adjusts a configuration of the at least one coil within the housing.

2. The charging device of claim 1, wherein the coil manipulator moves the at least one coil within the housing.

3. The charging device of claim 2, wherein the coil manipulator moves the at least one coil within the housing by translating a position of the at least one coil within the housing.

4. The charging device of claim 2, wherein the coil manipulator moves the at least one coil within the housing by rotating a position of the at least one coil within the housing.

5. The charging device of claim 1, further comprising:
at least one sensor that provides a sensor reading to a charging engine.

6. The charging device of claim 5, wherein the charging engine instructs the coil manipulator to adjust the configuration of the at least one coil based on the sensor reading.

7. The charging device of claim 5, wherein the sensor reading provides an indication of an efficiency with which the at least one coil is wirelessly transferring energy to the implantable medical device.

8. The charging device of claim 5, wherein the sensor reading provides an indication of a distance between the at least one coil and a receiver coil in the implantable medical device.

9. The charging device of claim 1, wherein the at least one coil is provided on a support substrate in the housing and wherein the coil manipulator adjusts the configuration of the at least one coil by moving the support substrate.

10. The charging device of claim 9, wherein the at least one coil comprises a wire mounted on the support substrate.

11. The charging device of claim 9, wherein the at least one coil comprises a conductive material printed on the support substrate.

12. The charging device of claim 1, wherein the at least one coil comprises a single coil.

13. The charging device of claim 1, wherein the at least one coil comprises an array of coils.

14. The charging device of claim 13, wherein the coil manipulator comprises one or more electrical switches that selectively activate and deactivate coils in the array of coils.

15. The charging device of claim 1, wherein the configuration of the at least one coil is adjusted to maximize charging efficiency between the at least one coil and the implantable medical device.

16. A system comprising:
a chargeable device having a receiver coil; and
a charging device comprising:
a charging coil that wirelessly transfers energy to the receiver coil; and
a coil manipulator that changes a configuration of the charging coil to improve an efficiency with which the energy is wirelessly transferred to the receiver coil during a charging session.

17. The system of claim 16, further comprising:
at least one sensor that provides a sensor reading to determine an efficiency with which the energy is wirelessly transferred to the receiver coil during the charging session.

18. The system of claim 17, wherein the at least one sensor comprises one or more of an electromagnetic sensor, an optical sensor, an acoustic sensor, a mechanical sensor, and a thermal sensor.

19. The system of claim 16, wherein the coil manipulator at least one of physically moves the charging coil or adjusts an RF field produced by the charging coil.

20. A system comprising:
a housing;
a charging coil contained within the housing; and
a coil manipulator that physically moves the charging coil in the housing.
